(19) United States
(12) Patent Application Publication
(10) Pub. No.: US 2023/0108890 A1
(43) Pub. Date: Apr. 6, 2023

(54) CELL CULTURE MANAGEMENT SYSTEM

(71) Applicant: Integriculture Inc., Tokyo (JP)

(72) Inventors: Ikko KAWASHIMA, Tokyo (JP); Yuki HANYU, Tokyo (JP)

(21) Appl. No.: 17/798,334

(22) PCT Filed: Feb. 10, 2020
(86) PCT No.: PCT/JP2020/005189

§ 371 Date: Aug. 9, 2022

(51) Int. Cl. G01N 33/483; G06Q 10/08

(52) U.S. Cl. CPC G01N 33/4833; G06Q 10/087

(57) ABSTRACT

The present invention provides cell culture management systems. A cell culture management system according to the present invention includes an output data server provided in a first cultural device for culturing a first kind of cells; a customer server provided in a second cultural device for culturing a second kind of cells; and a cell management server provided in a cell management compartment where the second kind of cells are stored, in which the output data server, the customer server, and the cell management server are each connected to and in bidirectional communication with a data management device.

### TECHNICAL FIELD

The present invention relates to cell culture management systems.

### BACKGROUND ART

Various kinds of cultural devices and approaches have been developed to grow internal organs or maintain their functions over a prolonged time. For example, WO2017/191691 discloses growth induction systems, growth induction control devices, growth induction control methods, and growth induction control programs with which growth can be induced without the need to add cytokines at different stages of growth.

### Problems to be Solved by the Invention

Nevertheless, no system has ever existed to manage such cell culture systems as a whole when they are individually installed.

Therefore, an object of the present invention is to provide novel cell culture management systems.

### Means to Solve the Problems

An aspect of the present invention is a cell culture management system including: a data management device; an output data server provided in a first cultural device for culturing a first kind of cells; a customer server provided in a second cultural device for culturing a second kind of cells; and a cell management server provided in a cell management compartment where the second kind of cells are stored, in which: the data management device is connected to and in bidirectional communication with each of the output data server, the customer server, and the cell management server; the output data server has: a transmitter for transmitting a first protocol for culturing the first kind of cells to the data management device; the customer server has: one or more transmitters for transmitting order information for obtaining the second kind of cells and location information of the second cultural device to the data management device; and a receiver for receiving a second protocol for culturing the second kind of cells from the data management device; the data management device has: a receiver for receiving the first protocol from the output data server; and a receiver for receiving the order information and the location information of the second cultural device from the customer server; and one or more memorizing units that memorize the first protocol received from the output data server, the second protocol, the order information and the location information of the second cultural device received from the customer server; and the cell management server has: a receiver for receiving the order information and the location information of the second bioreactor from the data management device. The output data server may have a receiver for receiving a third protocol for culturing a third kind of cells from the data management device. The customer server may have a transmitter for transmitting an estimated culture data obtained from a cell sensor disposed in the second cultural device to the data management device; and the data management device may have a receiver for receiving the estimated culture data transmitted from the customer server; and a memorizing unit that memorizes the estimated culture data received from the customer server. The cell management server may have a memorizing unit for memorizing inventory information for cells present in the cell management compartment; and a transmitter for transmitting the inventory information to the data management device; and the data management device may have a receiver for receiving the inventory information transmitted from the cell management server; and a memorizing unit for memorizing the inventory information that has been received.

Another aspect of the present invention is a cell culture management system including: an output data server provided in a first cultural device for culturing a first kind of cells; a customer server provided in a second cultural device for culturing a second kind of cells; and a cell management server provided in a cell management compartment where the second kind of cells are stored in which: the output data server, the customer server, and the cell management server are each connected to and in bidirectional communication with a data management device; the output data server has: a transmitter for transmitting a first protocol for culturing the first kind of cells to the data management device; the customer server has: one or more transmitters for transmitting order information for obtaining the second kind of cells and location information of the second cultural device to the data management device; and a receiver for receiving a second protocol for culturing the second kind of cells from the data management device; and the cell management server has: a receiver for receiving the order information and the location information of the second cultural device from the data management device. The output data server may have a receiver for receiving a third protocol for culturing a third kind of cells from the data management device. The customer server may have a transmitter for transmitting an estimated culture data obtained from a cell sensor disposed in the second cultural device to the data management device; and the data management device may have a receiver for receiving the estimated culture data transmitted from the customer server; and a memorizing unit that memorizes the estimated culture data received from the customer server. The cell management server may have a memorizing unit for memorizing inventory information for cells present in the cell management compartment; and a transmitter for transmitting the inventory information to the data management device; and the data management device may have a receiver for receiving the inventory information transmitted from the cell management server; and a memorizing unit for memorizing the inventory information that has been received.

### BRIEF DESCRIPTION OF THE DRAWINGS

FIG. 1 is a flow chart showing an operation of a cell culture system in a basic process, which is an embodiment according to the present invention;

FIG. 2 is a schematic diagram of a cell culture system in the basic process, which is an embodiment according to the present invention, in which broken lines indicate flows of information, and solid lines indicate flows of objects;

FIG. 3 is a schematic diagram of a cell culture system in novel protocol development, which is another embodiment according to the present invention, in which broken lines indicate flows of information, and solid lines indicate flows of objects;

FIG. 4 is a schematic diagram of a cell culture system in culture condition feedbacks, which is another embodiment according to the present invention, in which broken lines indicate flows of information, and solid lines indicate flows of objects; and

FIG. 5 is a schematic diagram of a cell culture system in inventory management, which is another embodiment according to the present invention, in which lines indicate flows of information, and solid lines indicate flows of objects.

### MODES FOR CARRYING OUT THE INVENTION

Preferred embodiments of the present invention are described in detail with reference to the accompanying drawing, but the present invention is not necessarily limited thereto. Objects, characteristics, advantages, and ideas of the present invention are apparent to a person skilled in the art from the description of the present specification, and the person skilled in the art can easily reproduce the present invention from the description of the present specification. Modes for carrying out the invention, specific examples thereof and so forth, which are described below, provide preferable embodiments of the present invention. They are described for the purpose of illustration or explanation, and thus the present invention is not limited thereto. It is apparent to a person skilled in the art that various alterations and modifications can be made on the basis of the description of the present specification within the spirit and the scope of the present invention disclosed in the present specification.

### ==Cell Culture Management System==

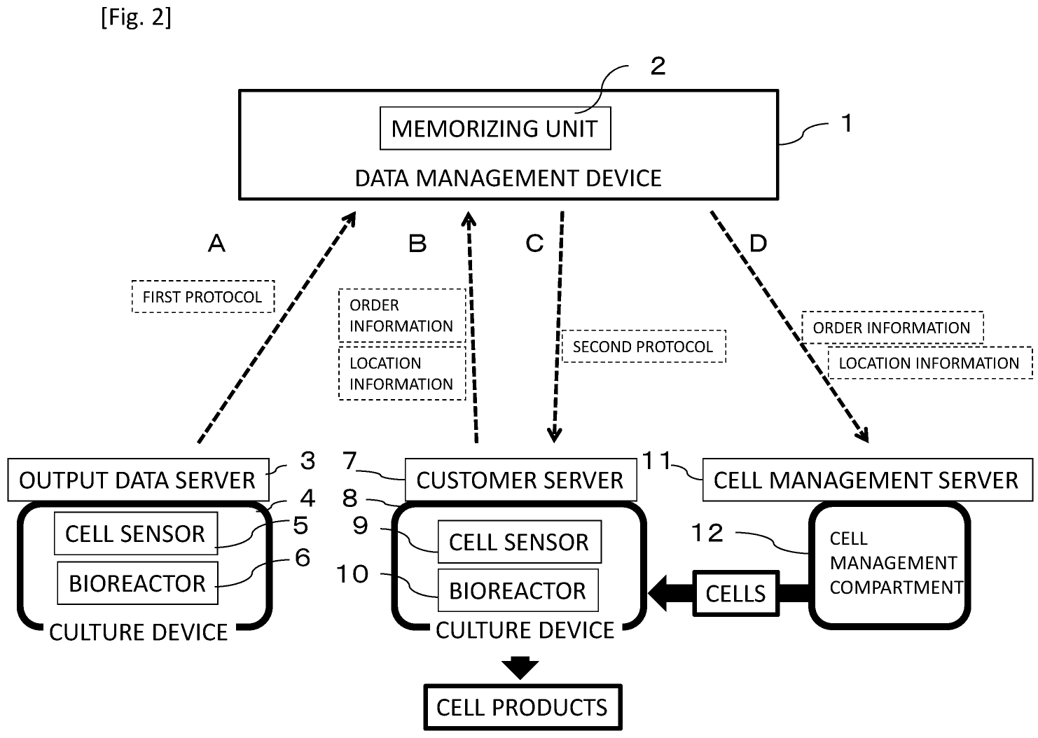

A cell culture management system according to an embodiment of the present invention includes an output data server provided in a first cultural device for culturing a first kind of cells; a customer server provided in a second cultural device for culturing a second kind of cells; and a cell management server provided in a cell management compartment where the second kind of cells are stored, in which the output data server, the customer server, and the cell management server are each connected to and in bidirectional communication with a data management device external to the system; the output data server has a transmitter for transmitting a first protocol for culturing the first kind of cells to the data management device; the customer server has one or more transmitters for transmitting order information for obtaining the second kind of cells and location information of the second cultural device to the data management device, and a receiver for receiving a second protocol for culturing the second kind of cells from the data management device; and the cell management server has a receiver for receiving the order information and the location information of a second bioreactor from the data management device. Here, the data management device has a receiver for receiving the first protocol from the output data server; a receiver for receiving the order information and the location information of the second cultural device from the customer server; and one or more memorizing units that memorize the first protocol received from the output data server, the second protocol, the order information, and the location information of the second bioreactor received from the customer server. A specific embodiment of this system is described below with reference to FIG. 2.

### <Data Management Device>

A data management device 1 is connected to and in bidirectional communication with one or more output data servers 3, one or more customer servers 7, and one or more cell management servers 11 via a network such as the Internet, a local area network (LAN), a wide area network (WAN), or a leased line. The data management device 1 has, at least, a receiver for receiving a protocol or protocols from the output data server(s) 3 and a receiver for receiving order information and location information of the (or each) cultural device 8 from the customer server(s) 7, and also has a memorizing unit that stores the received data. The received data are preferably stored in a database.

The data management device 1 may be external to the cell culture management system as in a cloud device, or it may be managed as a component of the cell culture management system.

The data management device 1 may be a simple device configured with a transceiver and the memorizing unit 2 only for holding data. Alternatively, it may have a data processor other than the transceiver and the memorizing unit 2 to perform various processing on the data.

### <Output Data Server>

The output data server(s) 3 is/are included in a cultural device (ore respective cultural devices) 4 for developing new protocols for culturing cells. The (or each) output data server 3 has a transmitter for transmitting a new protocol to the data management device 1. The protocol may include, for example, information for cell culture, such as the type of cell, culture conditions (e.g., the temperature and CO₂ concentration), the type of medium to be used, a reference value for a cell marker to be monitored over time, a reference value for the pH of a medium to be evaluated over time, and/or a reference value for a cell density to be monitored over time.

The cultural device(s) 4 is/are unlimited and any known cultural device may be used. For example, a cultural device 4 may consist of only an incubator for culturing cells; however, it may also include a bioreactor 6. The bioreactor 6 includes, for example, one or more culture chamber tanks for culturing cells and producing cell products, a control unit for regulating cell culture, and a memory for memorizing culture protocols including culture conditions. When two or more culture chambers are applied, each culture chamber may have an individual culture medium cycling mechanism to cycle the flow of the culture medium within the culture chamber, or a culture medium circulator may be included when the culture chambers are connected to each other to allow the culture medium to circulate between them. The control unit for regulating culture may regulate the flow rate of a culture medium and the direction of cycling a culture medium using the culture medium cycling mechanism.

The (or each) cultural device 4 preferably has a cell sensor 5 that can automatically detect the condition of the cell culture over time. The cell sensor 5 can preferably obtain physical, chemical, and biological characteristics and/or properties of cells, culture media or cell products in a culture chamber over time, including their weight, temperature, viscosity, color tone (absorbance), pH, sugar content, salinity, density, and type. The cell sensor 5 may be controlled by the control unit. The conditions detected using the cell sensor 5 may be stored in a memory.

Furthermore, the data management device 1 may have a transmitter that transmits culture protocols to the output data server(s) 3, while the (or each) output data server 3 may have a receiver that receives culture protocols from the data management device 1. Culture protocol creators may create an entirely new protocol, but having a way to receive protocols enables the creators to create a new protocol by changing an existing culture protocol.

### <Customer Server>

The customer server(s) 7 is/are included in a cultural device (or respective cultural devices) 8 for customers to culture cells. The cultural device(s) 8 may have a similar structure to a cultural device 4 related to an output data server 3. For example, a cultural device 8 may include a bioreactor 10 and a cell sensor 9 which are similar to those in a cultural device 4. Particularly, when a novel protocol is developed in a cultural device 4 related to an output data server 3, it is preferable that the cultural devices 4 and 8 are exactly of the same model in order to use identical conditions.

For customers, the culture's purposes are unlimited. For example, cells may be grown for food, medical or material use, or a substance may be formed by employing cells.

The (or each) customer server 7 has a transmitter for transmitting order information and location information of the local cultural device 8 to the data management device 1. The content of the order information is unlimited as long as the customer's purpose is explained. For example, when a kind of cells is needed, it may be the desired cell's name, or when a kind of cell products is needed, it may be the cell product's name Furthermore, besides the name of the desired cells or cell products, information that is often applied to order a commercial item may be included. For example, by including information on when the cells or cell products are required, it is feasible to estimate when the cells will be delivered from a cell management compartment. The location information of a given cultural device 8 denotes where the cultural device 8 in question is. That location can be recognized using, for example, its address.

The customer server(s) 7 also has/have a receiver for receiving a protocol or protocols for producing the desired kind of cells or cell products from the data management device 1. The protocols received from the data management device 1 may be stored in a memory included in a bioreactor.

In addition, the (or each) customer server 7 may have a transmitter for transmitting, to the data management device 1, estimated culture data including a measured value corresponding to a reference value for a protocol (e.g., reference values for pH and cell density) obtained over time from a cell sensor in the bioreactor while the cells are being cultured in a cultural device 8 or from a cell sensor at the end of the culture. Having such a way allows for the detection and management of variations when similar cell products are produced in two or more bioreactors. In this situation, the data management device may have a receiver for receiving the estimated culture data and a memorizing unit for memorizing the estimated culture data. The memorizing unit may either share an area with or be achieved separately from a memorizing unit for storing other data.

When two or more culture chambers are used in the bioreactor 10, the protocol may also include information on where culture cells are placed at the beginning (e.g., a culture chamber number in the bioreactor) and instructions for changing culture media cycling conditions between or among culture chambers over time.

### <Cell Management Server>

The cell management server(s) 11 is/are included in a cell management compartment (or respective cell management compartments) 12 where cells are stored and managed, and has/have a receiver for receiving, from the data management device 1, order information transmitted from customers using their local customer servers, and location information of their local cultural devices 8 for customers to culture the desired cells. Additionally, the (or each) cell management server 11 may receive a culture protocol or protocols as well as delivery item information, which are associated with the order information from the data management device 1. The delivery item information denotes an item or items to be delivered to a customer in response to an order from that customer and includes information regarding the materials needed for culture (e.g., consumables such as culture medium). Based on these pieces of information, it becomes feasible to deliver the item(s) requested by the customer to the place where the cultural device 8 is situated.

The (or each) cell management server 11 may have a transmitter for transmitting inventory information for different cells stored in a cell management compartment or the local cell management server 11 to the data management device 1. A cell administrator accessing the data management device 1 may refill a low or no inventory of cells by purchasing cells or growing the cells by himself.

The cell management compartment may include a warehouse, a refrigerator, a freezer, and/or a culture chamber for storing cells and distributing them when instructed to do so.

### ==Cell Culture Management Method==

How cells can be managed using the aforementioned cell culture management system is described in detail in terms of several embodiments used as examples.

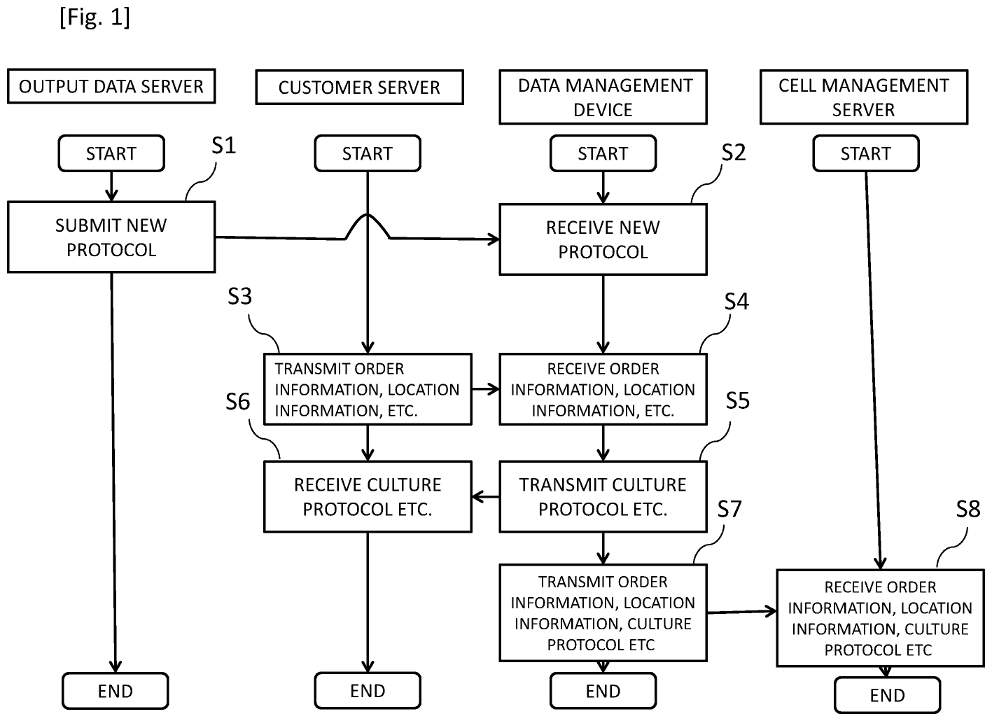

### (1) Basic Process (FIGS. 1 and 2)

A basic process in the cell culture management method using this management system is described using FIGS. 1 and 2.

When a culture protocol creator submits a culture protocol in which the names and IDs of cells and/or cell products are associated with a related cell culture method, to the data management device 1 via an output data server 3 (S1), the data management device 1 receives the data transmitted from the output data server 3 and stores it in a memorizing unit 2 (S2)(A). The cell culture method is developed by the culture protocol creator, and at that time, the culture conditions in his or her local bioreactor 6 detected by the local cell sensor 5 may be included in the culture protocol.

A customer places an order to the data management device 1 to obtain the desired cells using his or her local customer server 7. At that time, when the location information of a cultural device 8 where cells are produced and other information are transmitted to the data management device 1 with the order information (S3), the data management device 1 receives the data transmitted from the customer server 7 and stores the data in the memorizing unit 2 (S4)(B). The order information may be related to the cells that the customer wants to culture, or related to the cell products that the customer wants to produce using the cultured cells. Here, the information is preferably the name of the cells or cell products, but it may be an ID when the data management device manages the cultured cells, cell products, and/or culture protocols by their IDs and the customer is aware of the IDs.

When the data management device 1 receives the order from the customer server 7 and delivers a culture protocol for culturing cells (S5), the customer receives, through the customer server 7, the data transmitted from the data management device 1 (S6)(C). For this, the data management device 1 may automatically deliver a culture protocol matching the order information in response to its receipt, or the customer may select and download a suitable culture protocol from the data in the data management device 1.

The data management device 1 that has received the order from the customer delivers the order information from the customer, the location information of the customer's cultural device, and some other information (S7), and a cell management server 11 in a cell management compartment 12 receives the data from the data management device 1 (S8)(D). For receiving data by the cell management server 11, the cell management server 11 may, for example, retrieve the data from the data management device 1 at regular intervals, or an administrator of the cell management compartment 12 may access the data management device 1 to get the data at his or her convenient time. Alternatively, the data management device 1 that has received the order may automatically transmit the data to the cell management server 11, along with, for example, the location information of the customer's cultural device. The administrator of the cell management compartment 12 subsequently delivers the item(s) requested by the customer to the customer's cultural device 8 according to the received data.

The customer, using the delivered item(s) received from the cell management compartment 12, places the cells in the culture chamber of the bioreactor 10 according to the received culture protocol and initiates culture to grow the desired cells. Alternatively, cell products generated from the cell growth may be collected.

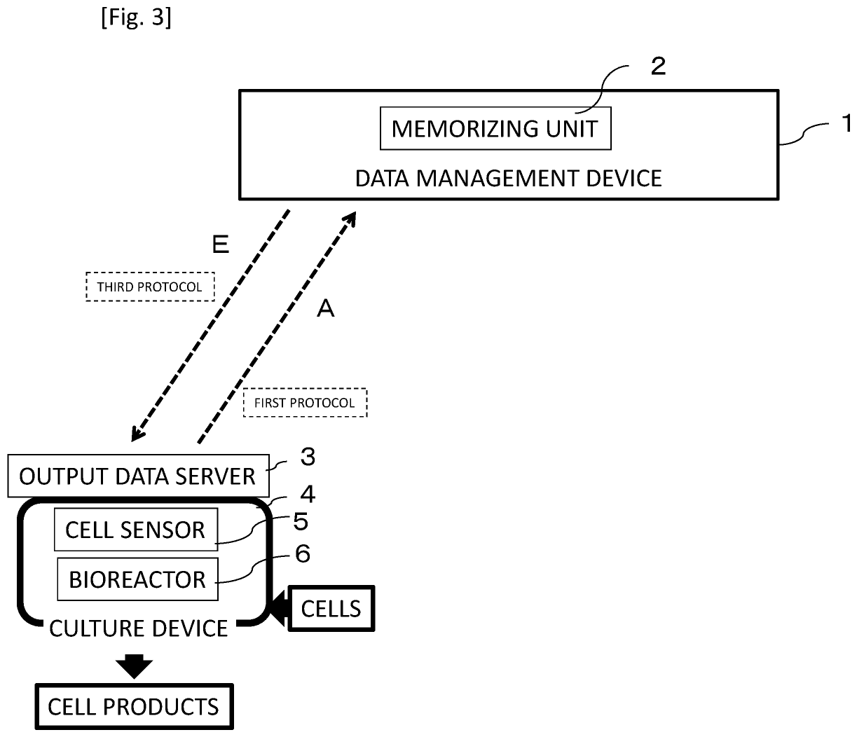

### (2) Creation of Novel Protocols (FIG. 3)

An embodiment in which an output data server 3 transmits a novel protocol to the data management device 1 is described with reference to FIG. 3.

A culture protocol creator can develop an entirely new protocol and submit it to the data management device 1 with a new management number (A). Alternatively, he or she may receive an existing protocol “a” from the data management device 1 through an output data server 3(E), investigate the protocol using a cultural device 4, develop a new protocol “b” from the protocol “a”, and then submit the protocol “b” to the data management device 1 with a new management number (A).

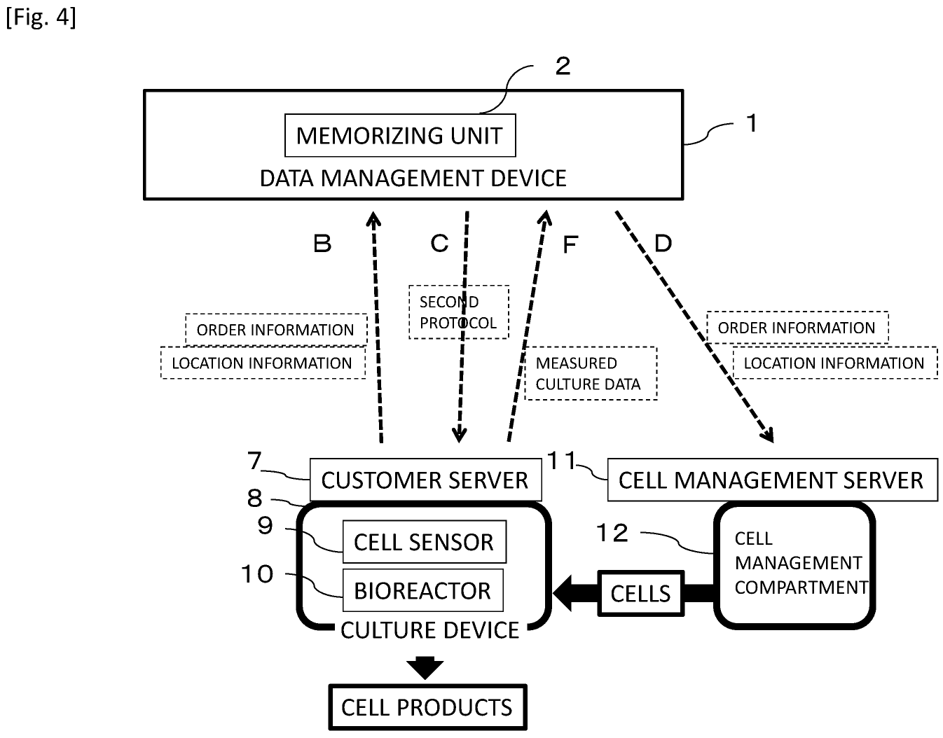

(3) Feedback from Customers (FIG. 4)

An embodiment in which a customer server 7 feeds back evaluated culture data to the data management device 1 is described with reference to FIG. 4.

A customer accesses the data management device 1 using his or her local customer server 7 and places an order. At that time, the order information and the location information of the cultural device 8 where the desired cells are cultured are transmitted to the data management device 1(B). The data management device 1 receives these pieces of information and memorizes them in the memorizing unit 2. In response to this, the customer server 7 receives a culture protocol for culturing the desired cells from the data management device 1(C).

When a cell management server 11 included in a cell management compartment 12 receives the order information from the customer, the location information of the customer's cultural device, or some other information transmitted from the data management device 1, an administrator of the cell management compartment 12 delivers the cells to the customer's cultural device 8 according to the received data (D).

The customer may load the received protocol into the bioreactor 10 and starts culturing the cells in a predefined culture chamber tank in the bioreactor 10. In doing so, the customer server 7 may transmit, to the data management device 1, estimated culture data including a measured value corresponding to a reference value for a protocol (e.g., reference values for pH and cell density) obtained over time from a cell sensor 9 in the cultural device 8 while the desired cells are being cultured in the cultural device 8 or from a cell sensor at the end of the culture of the desired cells (F). The data management device 1 receives the estimated culture data and may store it in the memorizing unit 2.

By managing such estimated culture data in the data management device 1, culture protocol creators can freely utilize the estimated culture data in developing protocols.

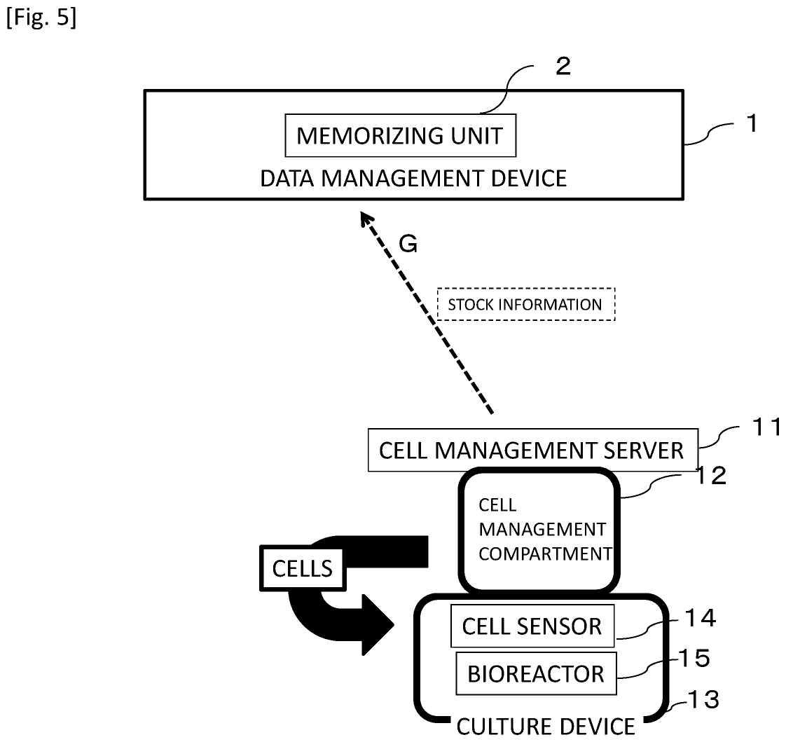

### (4) Inventory Management (FIG. 5)

An embodiment in which a cell management server 11 manages an inventory of cells is described with reference to FIG. 5.

The cell management server 11 may transmit cell inventory information to the data management device 1(G). This may be achieved by periodically uprating the cell inventory information. Alternatively, the inventory information may be uprated only when the content of the cell inventory information has been changed at the cell management server 11. The data management device 1 manages the inventory information as a database in the storage device 2. When a new piece of inventory information is transmitted from the cell management server 11, the data management device 1 receives it and updates the database in the storage device 2. The inventory information may include not only the inventory quantity, but also expiration date information and/or some other information.

An administrator of a cell management compartment 12 accesses the data management device 1 and delivers new cells into the cell management compartment 12 for the kinds of cells that are low or no longer in stock, or past their use-by date. Alternatively, to ensure cell inventory, cells are grown using a cultural device 13 included in the cell management compartment 12. The cultural device 13 may be similar to or different from a cultural device 4 of an output data server 3 or a cultural device 4 of a customer server 7. It is, however, preferable that the cultural device 13 is of the same type and has a cell sensor 14 and a bioreactor 15.

### ==Specific Example of Operation Using a Cell Culture Management System==

This section describes exemplified operations of the cell culture management system as far as customers are concerned. These embodiments enable customers to obtain a culture protocol and cells appropriate for producing desired cells or cell products using a specific cultural device by indicating desired kind of cells or cell products, and to produce cells or cell products with a high reproducibility by culturing cells with the obtained culture protocol.

### (1) For Customers Ordering Cells

Customers are given in advance a cultural device for culturing desired cells.

When a customer requires to culture cells, he or she accesses the data management device 1 using a customer server 7. The data management device 1 presents a list of cells that can be ordered and an order form as a database. The customer selects a kind of cells from the list, clicks on the name or ID of the cell or writes the name or ID of the cell; simultaneously, fills the address of a cultural device 8 where cells will be produced in the order form. Finally, the customer transmits these pieces of information to the data management device 1.

The customer may download a culture protocol associated with the ordered kind of cells. The culture protocol describes how to culture the cells of interest (e.g., type of medium, culture conditions, etc.).

Upon learning of the order from the data management device 1, an administrator of a cell management compartment 12 delivers to the customer an item or items associated with that kind of cells and necessary for their culture, and the customer receives the delivered item(s). The customer can culture the desired cells using the received item(s) in the cultural device according to the downloaded culture protocol.

### (2) For Customers Ordering Cell Products

Customers are given in advance a cultural device for culturing cells to generate desired cell products.

When a customer requires generate cell products, he or she accesses the data management device 1 using a customer server 7. The data management device 1 presents a list of cell products that can be generated and an order form as a database. The customer selects a kind of cell products from the list, clicks on the name or ID of the cell product or writes the name or ID of the cell product; simultaneously, fills the address of a cultural device 8 where cells will be produced in the order form. Finally, the customer transmits these pieces of information to the data management device 1.

The customer may download a culture protocol associated with the ordered kind of cell products. The culture protocol describes how to culture cells to generate the cell products of interest (e.g., type of cell, type of medium, culture conditions, etc.).

Upon learning of the order from the data management device 1, an administrator of a cell management compartment 12 delivers to the customer an item or items associated with that kind of cell products and necessary for cell culture for their generation, and the customer receives the delivered item(s). The customer can generate the desired cell products by culturing given cells using the received item(s) in the cultural device according to the downloaded culture protocol.

### DENOTATION OF SYMBOLS

1 data management device 1
2 memorizing unit
3 output data server
4 cultural device
5 cell sensor
6 bioreactor
7 customer server
8 cultural device
9 cell sensor
10 bioreactor
11 cell management server
12 cell management compartment
13 cultural device
14 cell sensor
15 bioreactor

### INDUSTRIAL APPLICABILITY

The present invention made it possible to provide novel cell culture management systems.

1. A cell culture management system comprising:
a data management device;
an output data server provided in a first cultural device for culturing a first kind of cells;
a customer server provided in a second cultural device for culturing a second kind of cells; and
a cell management server provided in a cell management compartment where the second kind of cells are stored, wherein:
the data management device is connected to and in bidirectional communication with each of the output data server, the customer server, and the cell management server;
the output data server comprises:
a transmitter for transmitting a first protocol for culturing the first kind of cells to the data management device;
the customer server comprises:
one or more transmitters for transmitting order information for obtaining the second kind of cells and location information of the second cultural device to the data management device; and
a receiver for receiving a second protocol for culturing the second kind of cells from the data management device;
the data management device comprises:
a receiver for receiving the first protocol from the output data server; and
a receiver for receiving the order information and the location information of the second cultural device from the customer server; and
one or more memorizing units that memorize the first protocol received from the output data server, the second protocol, the order information and the location information of the second cultural device received from the customer server; and
the cell management server comprises:
a receiver for receiving the order information and the location information of the second bioreactor from the data management device.

2. The cell culture management system according to claim 1, wherein the output data server comprises a receiver for receiving a third protocol for culturing a third kind of cells from the data management device.

3. The cell culture management system according to claim 1, wherein:
the customer server comprises a transmitter for transmitting an estimated culture data obtained from a cell sensor disposed in the second cultural device to the data management device; and
the data management device comprises:
a receiver for receiving the estimated culture data transmitted from the customer server; and
a memorizing unit that memorizes the estimated culture data received from the customer server.

4. The cell culture management system according to claim 1, wherein:
the cell management server comprises:
a memorizing unit for memorizing inventory information for cells present in the cell management compartment; and
a transmitter for transmitting the inventory information to the data management device; and the data management device comprises:
a receiver for receiving the inventory information transmitted from the cell management server; and
a memorizing unit for memorizing the inventory information that has been received.

5. A cell culture management system comprising:
an output data server provided in a first cultural device for culturing a first kind of cells;
a customer server provided in a second cultural device for culturing a second kind of cells; and
a cell management server provided in a cell management compartment where the second kind of cells are stored, wherein:
the output data server, the customer server, and the cell management server are each connected to and in bidirectional communication with a data management device;
the output data server comprises:
a transmitter for transmitting a first protocol for culturing the first kind of cells to the data management device;
the customer server comprises:
one or more transmitters for transmitting order information for obtaining the second kind of cells and location information of the second cultural device to the data management device; and
a receiver for receiving a second protocol for culturing the second kind of cells from the data management device; and
the cell management server comprises:
a receiver for receiving the order information and the location information of the second cultural device from the data management device.

6. The cell culture management system according to claim 5, wherein the output data server comprises a receiver for receiving a third protocol for culturing a third kind of cells from the data management device.

7. The cell culture management system according to claim 5, wherein:
the customer server comprises a transmitter for transmitting an estimated culture data obtained from a cell sensor disposed in the second cultural device to the data management device; and
the data management device comprises:
a receiver for receiving the estimated culture data transmitted from the customer server; and
a memorizing unit that memorizes the estimated culture data received from the customer server.

8. The cell culture management system according to claim 5, wherein:
the cell management server comprises:
a memorizing unit for memorizing inventory information for cells present in the cell management compartment; and
a transmitter for transmitting the inventory information to the data management device; and the data management device comprises:
a receiver for receiving the inventory information transmitted from the cell management server; and
a memorizing unit for memorizing the inventory information that has been received.
